(19) United States
(12) Patent Application Publication
(10) Pub. No.: US 2004/0225014 A1
(43) Pub. Date: Nov. 11, 2004

(54) Method for treating ocular hypertension and glaucoma

(75) Inventors: Tsuyoshi Habe, Sasayama-shi (JP); Takashi Sekida, Nishinomiya-shi (JP)

Correspondence Address: SUGHRUE MION, PLLC, 2100 PENNSYLVANIA AVENUE, N.W., SUITE 800, WASHINGTON, DC, 20037, US

(73) Assignee: SUCAMPO AG

(21) Appl. No.: 10/776,610

(22) Filed: Feb. 12, 2004

Related U.S. Application Data
(60) Provisional application No. 60/447,299, filed on Feb. 14, 2003.

(51) Int. Cl. A61K 31/557

(52) U.S. Cl. 514/573

(57) ABSTRACT

The present invention provides a method for treating ocular hypertension and glaucoma, using a 15-keto-prostaglandin compound having a ring structure at the end of ω chain. The method of the present invention induces substantially no hair growth, which is a known side effect of conventional prostaglandin ophthalmic compounds such as latanoprost.

### CROSS-REFERENCE TO RELATED APPLICATION(S)

This application claims the benefit of provisional application No. 60/447,299 filed Feb. 14, 2003, the contents of which is incorporated herein by reference.

### TECHNICAL FIELD

The present invention relates to a method for treating ocular hypertension and glaucoma, which induces substantially no hair growth.

### BACKGROUND ART

Prostaglandins (hereinafter, referred to as PG(s)) are members of class of organic carboxylic acids, which are contained in tissues or organs of human or other mammals, and exhibit a wide range of physiological activity. PGs found in nature (primary PGs) generally have a prostanoic acid skeleton as shown in the formula (A):

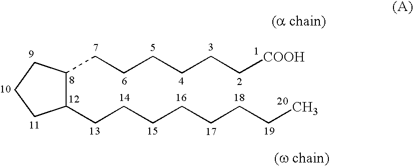

On the other hand, some of synthetic analogues of primary PGs have modified skeletons. The primary PGs are classified into PGAs, PGBs, PGCs, PGDs, PGEs, PGFs, PGGs, PGHs, PGIs and PGJs according to the structure of the five-membered ring moiety, and further classified into the following three types by the number and position of the unsaturated bond at the carbon chain moiety:

Subscript 1: 13,14-unsaturated-15-OH

Subscript 2: 5,6- and 13,14-diunsaturated-15-OH

Subscript 3: 5,6-, 13,14-,and 17,18-triunsaturated-15-OH.

Further, the PGFs are classified, according to the configuration of the hydroxyl group at the 9-position, into α type (the hydroxyl group is of an α-configuration) and β type (the hydroxyl group is of a β-configuration).

PGE₁ and PGE₂ and PGE₃ are known to have vasodilation, hypotension, gastric secretion decreasing, intestinal tract movement enhancement, uterine contraction, diuretic, bronchodilation and anti ulcer activities. PGF_{1α}, PGF_{2α} and PGF_{3α} have been known to have hypertension, vasoconstriction, intestinal tract movement enhancement, uterine contraction, lutein body atrophy and bronchoconstriction activities.

Some 15-keto (i.e., having oxo at the 15-position instead of hydroxy)-PGs and 13,14-dihydro (i.e., having single bond between the 13 and 14-position)-15-keto-PGs are known as the substances naturally produced by the action of enzymes during the metabolism of primary PGs. It is also known that some 15-keto-PG compounds have IOP reducing effects and are effective for the treatment of ocular hypertension and glaucoma (U.S. Pat. Nos. 5,001,153, 5,151,444, 5,166,178, 5,212,200 and 6,458,836, all of which are incorporated herein by reference).

It is reported that various prostaglandin compounds having hydroxy (—OH) at the 15-position induce hair growth. For example, “Xalatan®”, “Lumigan®” and “Travatan®” eye drops that have been launched in the U.S.A. as a remedy for ocular hypertension and glaucoma contain, as its active ingredient, latanoprost, that is 13,14-dihydro-17-phenyl-18,19,20-trinor-PGF_{2α} isopropyl ester; bimatoprost, that is 17-phenyl 18,19,20-trinor PGF_{2α}-N-ethylamide; and travoprost, that is 16-(3-trifluoromethylphenoxy)-17,18,19,20-tetranor PGF_{2α} isopropyl ester, respectively, and induce eyelash hair growth, i.e. increase length, thickness, pigmentation and number of eyelashes (2002 Physician's Desk Reference, the reference is herein incorporated by reference).

It is also reported that “Rescula®” eye drops, that is prostaglandin compound having oxo (═O) at the 15-position; 13,14-dihydro-15-keto-20-ethyl-PGF_{2α} isopropylester, increases length of eyelashes (Rescula® package insert).

In this way, it has been considered to be difficult to achieve the IOP reduction using prostaglandin compounds without side effect such as hair growth.

### SUMMARY OF THE INVENTION

The present inventors conducted an intensive study and found that surprisingly 15-keto-prostaglandin compounds having a ring structure at the end of ω-chain possessed less hair growth activity, which have resulted in the completion of the present invention.

Namely, the present invention relates to a method for treating ocular hypertension and glaucoma, which comprises administration of a topical ophthalmic composition comprising as an active ingredient thereof 15-keto-prostaglandin compound having a ring structure at the end of ω chain, provided that the method induces substantially no hair growth.

The present invention further relates to a topical ophthalmic composition for treating ocular hypertension and glaucoma, which comprises as an active ingredient thereof 15-keto-prostaglandin compound having a ring structure at the end of ω chain, provided that the composition induces substantially no hair growth.

Furthermore, the present invention relates to a use of 15-keto-prostaglandin compound having a ring structure at the end of ω chain for manufacturing a topical ophthalmic composition for treating ocular hypertension and glaucoma, provided that the composition induces substantially no hair growth.

### DETAILED DESCRIPTION

In the present invention, the “15-keto-prostaglandin compound” (hereinafter, referred to as “15-keto-PG compound”) may include any of derivatives or analogs (including substituted derivatives) of a compound having an oxo group at 15-position of the prostanoic acid skeleton instead of the hydroxy group, irrespective of the configuration of the five-membered ring, the number of double bonds, presence or absence of a substituent, or any other modification in the α or ω chain.

The nomenclature of the 15-keto-PG compounds used herein is based on the numbering system of the prostanoic acid represented in the above formula (A).

A preferred compound used in the present invention is represented by the formula (I):

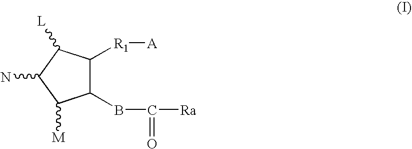

wherein L, M and N are hydrogen, hydroxy, halogen, lower alkyl, hydroxy(lower)alkyl, lower alkanoyloxy or oxo, wherein at least one of L and M is a group other than hydrogen, and the five-membered ring may have at least one double bond;

A is —CH₃, —CH₂OH, —COCH₂OH, —COOH or a functional derivative thereof;

B is —CH₂—CH₂—, —CH═CH— or —C≡C—;

R₁ is a saturated or unsaturated bivalent lower or medium aliphatic hydrocarbon, which is unsubstituted or substituted with halogen, alkyl, hydroxy, oxo, aryl or heterocyclic group, and at least one of carbon atom in the aliphatic hydrocarbon is optionally substituted by oxygen, nitrogen or sulfur; and

Ra is a saturated or unsaturated lower or medium aliphatic hydrocarbon, which is substituted at the end by cyclo(lower)alkyl, cyclo(lower)alkyloxy, aryl, aryloxy, heterocyclic group or hetrocyclic-oxy group, wherein the aliphatic hydrocarbon is optionally substituted by halogen, oxo, hydroxy, lower alkyl, lower alkoxy, lower alkanoyloxy.

A group of particularly preferable compounds among the above-described compounds is represented by the formula (II):

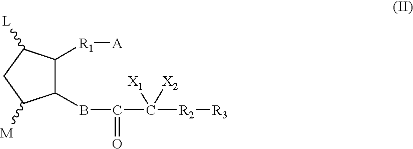

wherein L and M are hydrogen, hydroxy, halogen, lower alkyl, hydroxy(lower)alkyl, lower alkanoyloxy or oxo, wherein at least one of L and M is a group other than hydrogen, and the five-membered ring may have at least one double bond;

A is —CH₃, —CH₂OH, —COCH₂OH, —COOH or a functional derivative thereof;

B is —CH₂—CH₂—, —CH═CH—, —C≡C—;

X₁ and X₂ are hydrogen, lower alkyl, or halogen;

R₁ is a saturated or unsaturated bivalent lower or medium aliphatic hydrocarbon, which is unsubstituted or substituted with halogen, alkyl, hydroxy, oxo, aryl or heterocyclic group, and at least one of carbon atom in the aliphatic hydrocarbon is optionally substituted by oxygen, nitrogen or sulfur; and

R₂ is a single bond or lower alkylene; and

R₃ is cyclo(lower)alkyl, cyclo(lower)alkyloxy, aryl, aryloxy, heterocyclic group or heterocyclic-oxy group.

In the above formula, the term “unsaturated” in the definitions for R₁ and Ra is intended to include at least one or more double bonds and/or triple bonds that are isolatedly, separately or serially present between carbon atoms of the main and/or side chains. According to the usual nomenclature, an unsaturated bond between two serial positions is represented by denoting the lower number of the two positions, and an unsaturated bond between two distal positions is represented by denoting both of the positions.

The term “lower or medium aliphatic hydrocarbon” refers to a straight or branched chain hydrocarbon group having 1 to 14 carbon atoms (for a side chain, 1 to 3 carbon atoms are preferable) and preferably 1 to 10, especially 6 to 10 carbon atoms for R₁ and 1 to 10, especially 1 to 8 carbon atoms for Rₐ.

The term “halogen” covers fluorine, chlorine, bromine and iodine.

The term “lower” throughout the specification is intended to include a group having 1 to 6 carbon atoms unless otherwise specified.

The term “lower alkyl” refers to a straight or branched chain saturated hydrocarbon group containing 1 to 6 carbon atoms and includes, for example, methyl, ethyl, propyl, isopropyl, butyl, isobutyl, t-butyl, pentyl and hexyl.

The term “lower alkoxy” refers to a group of lower alkyl-O—, wherein lower alkyl is as defined above.

The term “hydroxy(lower)alkyl” refers to a lower alkyl as defined above which is substituted with at least one hydroxy group such as hydroxymethyl, 1-hydroxyethyl, 2-hydroxyethyl and 1-methyl-1-hydroxyethyl.

The term “lower alkanoyloxy” refers to a group represented by the formula RCO—O—, wherein RCO— is an acyl group formed by oxidation of a lower alkyl group as defined above, such as acetyl.

The term “cyclo(lower)alkyl” refers to a cyclic group formed by cyclization of a lower alkyl group as defined above but contains three or more carbon atoms, and includes, for example, cyclopropyl, cyclobutyl, cyclopentyl and cyclohexyl.

The term “cyclo(lower)alkyloxy” refers to the group of cyclo(lower)alkyl-O—, wherein cyclo(lower)alkyl is as defined above.

The term “aryl” may include unsubstituted or substituted aromatic hydrocarbon rings (preferably monocyclic groups), for example, phenyl, tolyl, xylyl. Examples of the substituents are halogen atom and halo(lower)alkyl, wherein halogen atom and lower alkyl are as defined above.

The term “aryloxy” refers to a group represented by the formula ArO—, wherein Ar is aryl as defined above.

The term “heterocyclic group” may include mono- to tri-cyclic, preferably monocyclic heterocyclic group which is 5 to 14, preferably 5 to 10 membered ring having optionally substituted carbon atom and 1 to 4, preferably 1 to 3 of 1 or 2 type of hetero atoms selected from nitrogen atom, oxygen atom and sulfur atom. Examples of the heterocyclic group include furyl, thienyl, pyrrolyl, oxazolyl, isoxazolyl, thiazolyl, isothiazolyl, imidazolyl, pyrazolyl, furazanyl, pyranyl, pyridyl, pyridazinyl, pyrimidyl, pyrazinyl, 2-pyrrolinyl, pyrrolidinyl, 2-imidazolinyl, imidazolidinyl, 2-pyrazolinyl, pyrazolidinyl, piperidino, piperazinyl, morpholino, indolyl, benzothienyl, quinolyl, isoquinolyl, purinyl, quinazolinyl, carbazolyl, acridinyl, phenanthridinyl, benzimidazolyl, benzimidazolinyl, benzothiazolyl, phenothiazinyl. Examples of the substituent in this case include halogen, and halogen substituted lower alkyl group, wherein halogen atom and lower alkyl group are as described above.

The term “heterocyclic-oxy group” means a group represented by the formula HcO—, wherein Hc is a heterocyclic group as described above.

The term “functional derivative” of A includes salts (preferably pharmaceutically acceptable salts), ethers, esters and amides.

Suitable “pharmaceutically acceptable salts” include conventionally used non-toxic salts, for example a salt with an inorganic base such as an alkali metal salt (such as sodium salt and potassium salt), an alkaline earth metal salt (such as calcium salt and magnesium salt), an ammonium salt; or a salt with an organic base, for example, an amine salt (such as methylamine salt, dimethylamine salt, cyclohexylamine salt, benzylamine salt, piperidine salt, ethylenediamine salt, ethanolamine salt, diethanolamine salt, triethanolamine salt, tris(hydroxymethylamino)ethane salt, monomethyl-monoethanolamine salt, procaine salt and caffeine salt), a basic amino acid salt (such as arginine salt and lysine salt), tetraalkyl ammonium salt and the like. These salts may be prepared by a conventional process, for example from the corresponding acid and base or by salt interchange.

Examples of the ethers include alkyl ethers, for example, lower alkyl ethers such as methyl ether, ethyl ether, propyl ether, isopropyl ether, butyl ether, isobutyl ether, t-butyl ether, pentyl ether and 1-cyclopropyl ethyl ether; and medium or higher alkyl ethers such as octyl ether, diethylhexyl ether, lauryl ether and cetyl ether; unsaturated ethers such as oleyl ether and linolenyl ether; lower alkenyl ethers such as vinyl ether, allyl ether; lower alkynyl ethers such as ethynyl ether and propynyl ether; hydroxy(lower)alkyl ethers such as hydroxyethyl ether and hydroxyisopropyl ether; lower alkoxy (lower)alkyl ethers such as methoxymethyl ether and 1-methoxyethyl ether; optionally substituted aryl ethers such as phenyl ether, tosyl ether, t-butylphenyl ether, salicyl ether, 3,4-di-methoxyphenyl ether and benzamidophenyl ether; and aryl(lower)alkyl ethers such as benzyl ether, trityl ether and benzhydryl ether.

Examples of the esters include aliphatic esters, for example, lower alkyl esters such as methyl ester, ethyl ester, propyl ester, isopropyl ester, butyl ester, isobutyl ester, t-butyl ester, pentyl ester and 1-cyclopropylethyl ester; lower alkenyl esters such as vinyl ester and allyl ester; lower alkynyl esters such as ethynyl ester and propynyl ester; hydroxy(lower)alkyl ester such as hydroxyethyl ester; lower alkoxy (lower) alkyl esters such as methoxymethyl ester and 1-methoxyethyl ester; and optionally substituted aryl esters such as, for example, phenyl ester, tolyl ester, t-butylphenyl ester, salicyl ester, 3,4-di-methoxyphenyl ester and benzamidophenyl ester; and aryl(lower)alkyl ester such as benzyl ester, trityl ester and benzhydryl ester.

The amide of A mean a group represented by the formula —CONR′R″, wherein each of R′ and R″ is hydrogen atom, lower alkyl, aryl, alkyl- or aryl-sulfonyl, lower alkenyl and lower alkynyl, and include for example lower alkyl amides such as methylamide, ethylamide, dimethylamide and diethylamide; arylamides such as anilide and toluidide; and alkyl- or aryl-sulfonylamides such as methylsulfonylamide, ethylsulfonyl-amide and tolylsulfonylamide.

Preferred examples of L and M include hydroxy which has a 5-membered ring structure of, so called, PGF type.

Preferred example of A is —COOH, its pharmaceutically acceptable salt, ester or amide thereof.

Preferred example of B is —CH₂—CH₂—, which provide the structure of so-called, 13,14-dihydro type.

Preferred example of X₁ and X₂ is hydrogen, or that at least one of them is halogen, more preferably, both of them are halogen, especially, fluorine that provides a structure of, so called 16,16-difluoro type.

Preferred R₁ is a hydrocarbon containing 1-10 carbon atoms, preferably, 6-10 carbon atoms. Further, at least one of carbon atom in the aliphatic hydrocarbon is optionally substituted by oxygen, nitrogen or sulfur.

Examples of R₁ include, for example, the following groups:

—CH₂—CH₂—CH₂—CH₂—CH₂—CH₂—,

—CH₂—CH═CH—CH₂—CH₂—CH₂—,

—CH₂—CH₂—CH₂—CH₂—CH═CH—,

—CH₂—C≡C—CH₂—CH₂—CH₂—,

—CH₂—CH₂—CH₂—CH₂—CH(CH₃)—CH₂—,

—CH₂—CH₂—CH₂—CH₂—O—CH₂—,

—CH₂—CH═CH—CH₂—O—CH₂—,

—CH₂—C≡C—CH₂—O—CH₂—,

—CH₂—CH₂—CH₂—CH₂—CH₂—CH₂—CH₂—,

—CH₂—CH═CH—CH₂—CH₂—CH₂—CH₂—,

—CH₂—CH₂—CH₂—CH₂—CH₂—CH═CH—,

—CH₂—C≡C—CH₂—CH₂—CH₂—CH₂—,

—CH₂—CH₂—CH₂—CH₂—CH₂—CH(CH₃)—CH₂—,

—CH₂—CH₂—CH₂—CH₂—CH₂—CH₂—CH₂—CH₂—,

—CH₂—CH═CH—CH₂—CH₂—CH₂—CH₂—CH₂—,

—CH₂—CH₂—CH₂—CH₂—CH₂—CH₂—CH═CH—,

—CH₂—C≡C—CH₂—CH₂—CH₂—CH₂—CH₂—,

—CH₂—CH₂—CH₂—CH₂—CH₂—CH₂—CH(CH₃)—CH₂—.

Preferred Ra is a hydrocarbon containing 1-10 carbon atoms, more preferably, 1-8 carbon atoms which is substituted by aryl or aryloxy at the end.

The configuration of the ring and the α- and/or ω chains in the above formula (I) and (II) may be the same as or different from that of the primary PGs. However, the present invention also includes a mixture of a compound having a primary type configuration and a compound of a non-primary type configuration.

The typical example of the present compound is a 13,14-dihydro-15-keto-17-phenyl-18,19,20-trinor-prostaglandin F compound and its derivative or analogue.

The 15-keto-PG compound of the present invention may be in the keto-hemiacetal equilibrium by formation of a hemiacetal between hydroxy at position 11 and oxo at position 15.

If such tautomeric isomers as above are present, the proportion of both tautomeric isomers varies with the structure of the rest of the molecule or the kind of the substituent present. Sometimes one isomer may predominantly be present in comparison with the other. However, it is to be appreciated that the 15-keto-PG compounds used in the invention include both isomers. Further, while the compounds used in the invention may be represented by a structure formula or name based on keto-type regardless of the presence or absence of the isomers, it is to be noted that such structure or name does not intend to exclude the hemiacetal type compound.

In the present invention, any of isomers such as the individual tautomeric isomers, the mixture thereof, or optical isomers, the mixture thereof, a racemic mixture, and other steric isomers may be used in the same purpose.

Some of the compounds used in the present invention may be prepared by the method disclosed in U.S. Pat. Nos. 5,073,569, 5,166,174, 5,221,763, 5,212,324 and 5,739,161 and U.S. patent application Ser. No. 09011218 (these cited references are herein incorporated by reference).

The term “treatment” or “treating” used herein includes any means of control such as prevention, care, relief of the condition, attenuation of the condition, arrest of progression and the like.

The phrase “induce substantially no hair growth” means the hair growth induced by the instant invention is far less than that induced by a known prostaglandin preparation for treating ocular hypertension and glaucoma such as latanoprost. The phrase does not mean that the compound of the present invention inhibits naturally occurring hair growth.

The present compound is applied by means of eye local administration, or administering an ophthalmic composition such as eye drop or eye ointment comprising said compound as an active ingredient.

The present composition may be manufactured according to any of the conventional methods. The form includes all the formulations for eye local administration used in the ophthalmic field.

The eye drops may be prepared by dissolving the active ingredients in a sterile aqueous solution such as saline, buffering solution or the like, or by combining powder compositions to be dissolved before use.

The eye drops may comprise an osmolarity modifier. The osmolarity modifiers may be any compound as far as they are ordinarily used in the ophthalmic field and the examples may include, but not limited thereto, sodium chloride, potassium chloride, calcium chloride, sodium bicarbonate, sodium carbonate, magnesium sulfate, sodium hydrogen phosphate, sodium dihydrogen phosphate, dipotassium hydrogen phosphate, boric acid, borax, sodium hydroxide, hydrochloric acid, mannitol, isosorbitol, propylene glycol, glucose and glycerine.

Further, additives ordinarily used in the ophthalmic field may be added to the present composition as desired. Such additives include, for example, buffer agent, e.g., boric acid, sodium monohydrogen phosphate and sodium dihydrogen phosphate; preservatives e.g. benzalkonium chloride, benzethonium chloride and chlorobutanol; thickeners e.g., saccharide such as lactose, mannitol and maltose, hyaluronic acid or its salt such as sodium hyaluronate and potassium hyaluronate, mucopolysaccharide such as chondroitin sulfate, sodium polyacrylate, carboxyvinyl polymer and crosslinked polyacrylate.

The eye ointments may be prepared by mixing the active ingredient into a base component conventionally used for known eye ointments under a sterile condition. Examples of the eye ointment bases may include, but not limited to, oil base such as vaseline, liquid paraffin, polyethylene, selen 50, plastibase, macrogol or a combination thereof; emulsion base having oil phase and water phase emulsified with surfactant; and water soluble base such as hydroxypropylmethylcellulose, carboxypropyl methylcellulose and polyethylene glycol. Further, in order to increase the hydrophilicity, a surface-active agent can be added to the composition. The eye ointment may also contain the above-mentioned additives such as the preservatives and the like, if desired.

The present composition may be formulated as a sterile unit dose type composition containing no preservatives.

The dose of the active ingredients of the composition used herein may vary according to the compound to be used, the type of the subject, age, weight, and symptom to be treated, desirable therapeutic effect, administration route, administration volume and period for treatment. Although suitable concentration may be chosen as desired, ordinarily, in the case of using the formulation of eye drops for an adult, the formulation containing 0.00001%-10%, preferably 0.0001%-5%, more preferably 0.001%-1% of the active ingredient can be instilled 1-6 times, preferably 1-2 times per day. The most preferable concentration of the active ingredient is 0.001%-0.05% and especially, 0.001%-0.03%.

In the case of using the formulation of eye ointments, the formulation containing 0.00001%-10%, preferably 0.0001%-5%, more preferably 0.001%-1% of the active ingredient can be applied, 1-6 times, preferably 1-2 times per day. The most preferable concentration of the active ingredient is 0.001%-0.05%, and especially, 0.001%-0.03%

According to the present invention, the above-described composition may be used for at least 2 weeks, preferably 3 month, more preferably 6 months, up to a time period as long as required, with inducing substantially no hair growth.

The present formulations may contain a single active ingredient or a combination of two or more active ingredients. In a combination of plural active ingredients, their respective contents may be suitably increased or decreased in consideration of their therapeutic effects and safety.

Further, the present formulations may suitably contain other pharmaceutically active ingredients, as far as they are not contrary to the objects of the present invention.

The present invention will be described in detail with reference to the following example, which, however, is not intended to limit the scope of the present invention.

### EXAMPLE

1) Test Method

Male C3H/HeN mice of 7 weeks old (seven groups; three mice/group) were used. Following 1-week acclimation, hair on the back of mice was shaved. Two days after the shaving, applications of test compound (13,14-dihydro-15-keto-17-phenyl-18,19,20-trinor-PGF_{2α} isopropyl ester), latanoprost(13,14-dihydro-17-phenyl-18,19,20-trinor-PGF_{2α} isopropyl ester), and vehicle (70 w/w % EtOH) were initiated (Day 1). 0.005%, 0.01% or 0.03% of test compound, 0.005%, 0.01% or 0.03% of latanoprost, or vehicle were applied to the shaved area of the mice at a volume of 0.1 mL once daily for 30 days. The hair growth was scored according to the following criteria.

### −: No hair growth observed

±: Hair growth observed; less than 10% of shaving area

+: Hair growth observed; 10-30% of shaving area

++: Hair growth observed; 30-50% of shaving area

+++: Hair growth observed; more than 50% of shaving area

2) Result

Scores of hair growth were shown in FIG. 1. Vehicle and Test compound at any concentration tested showed no effects on the hair growth. On the other hand, latanoprost exhibited remarkable hair growth stimulation.

These results indicate that hair growth activity of 15-keto-prostaglandin compound having a ring structure at the end of ω chain of the present invention significantly attenuates compared to the conventional PGs.

TABLE 1
Scores of hair growth

| | Animal | Days after first application | | | | | | | |
|---|---|---|---|---|---|---|---|---|---|
| | Num. | 19 | 22 | 23 | 24 | 25 | 26 | 29 | 30 |
| Group 1 | 11 | − | − | − | − | − | − | − | − |
| Vehicle¹⁾ | 12 | − | − | − | − | − | − | − | − |
| | 13 | − | − | − | − | − | − | − | − |
| Group 2 | 21 | − | − | − | − | − | − | − | − |
| 0.005% | 22 | − | − | − | − | − | − | − | − |
| Test compound | 23 | − | − | − | − | − | − | − | − |
| Group 3 | 31 | − | − | − | − | − | − | − | − |
| 0.01% | 32 | − | − | − | − | − | − | − | − |
| Test compound | 33 | − | − | − | − | − | − | − | − |
| Group 4 | 41 | − | − | − | − | − | − | − | − |
| 0.03% | 42 | − | − | − | − | − | − | − | − |
| Test compound | 43 | − | − | − | − | − | − | − | − |
| Group 5 | 51 | − | ± | + | + | ++ | ++ | ++ | +++ |
| 0.005% | 52 | − | − | ± | ± | ± | ± | + | ++ |
| Latanoprost | 53 | ± | ± | ± | ± | + | ++ | ++ | ++ |
| Group 6 | 61 | − | − | ± | + | + | + | ++ | ++ |
| 0.01% | 62 | − | − | − | ± | ± | ± | + | ++ |
| Latanoprost | 63 | − | − | ± | ± | ± | ± | + | + |
| Group 7 | 71 | − | ± | ± | ± | ± | ± | + | ++ |
| 0.03% | 72 | − | − | ± | ± | ± | + | + | + |
| Latanoprost | 73 | − | ± | ± | + | + | + | ++ | ++ |

¹⁾Vehicle: 70 w/w % aqueous ethanol

What is claimed is:

1. A method for treating ocular hypertension and glaucoma, which comprises administration of a topical ophthalmic composition comprising as an active ingredient thereof 15-keto-prostaglandin compound having a ring structure at the end of ω chain, to a subject in need of said treatment, provided that said method induces substantially no hair growth.

2. The method as described in claim 1, wherein said 15-keto-prostaglandin compound is a compound represented by the following general formula (I): 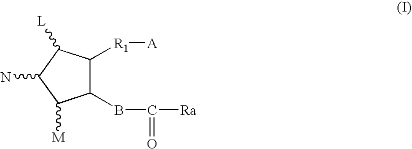
wherein L, M and N are hydrogen, hydroxy, halogen, lower alkyl, hydroxy(lower)alkyl, lower alkanoyloxy or oxo, wherein at least one of L and M is a group other than hydrogen, and the five-membered ring may have at least one double bond;
A is —CH₃, —CH₂OH, —COCH₂OH, —COOH or a functional derivative thereof;
B is —CH₂—CH₂—, —CH═CH— or —C≡C—;
R₁ is a saturated or unsaturated bivalent lower or medium aliphatic hydrocarbon, which is unsubstituted or substituted with halogen, alkyl, hydroxy, oxo, aryl or heterocyclic group, and at least one of carbon atom in the aliphatic hydrocarbon is optionally substituted by oxygen, nitrogen or sulfur; and
Ra is a saturated or unsaturated lower or medium aliphatic hydrocarbon, which is substituted at the end by cyclo(lower)alkyl, cyclo(lower)alkyloxy, aryl, aryloxy, heterocyclic group or hetrocyclic-oxy group, wherein the aliphatic hydrocarbon is optionally substituted by halogen, oxo, hydroxy, lower alkyl, lower alkoxy, lower alkanoyloxy.

3. The method as described in claim 1, wherein said 15-keto-prostaglandin compound is a 13,14-dihydro-15-keto-prostaglandin compound.

4. The method as described in claim 1, wherein said 15-keto-prostaglandin compound is a 13,14-dihydro-15-keto-17-phenyl-18,19,20-trinor-prostaglandin compound.

5. The method as described in claim 1, wherein said 15-keto-prostaglandin compound is 13,14-dihydro-15-keto-17-phenyl-18,19,20-trinor-PGF_{2α} isopropyl ester.
